(19) United States
(12) Patent Application Publication
(10) Pub. No.: US 2015/0366678 A1
(43) Pub. Date: Dec. 24, 2015

(54) MODULAR FOREARM

(71) Applicant: Fillauer LLC, Chattanooga, TN (US)

(72) Inventors: Steven Edwards, Chattanooga, TN (US); Samuel Hale, Ooltewah, TN (US); Karl Michael Fillauer, Hixson, TN (US)

(73) Assignee: FILLAUER LLC, Chattanooga, TN (US)

(21) Appl. No.: 14/744,968

(22) Filed: Jun. 19, 2015

Related U.S. Application Data
(60) Provisional application No. 62/015,034, filed on Jun. 20, 2014.

(51) Int. Cl. A61F 2/58

(52) U.S. Cl. CPC A61F 2/588; A61F 2/585; A61F 2002/5007

(57) ABSTRACT

A prosthetic device including a proximal ring having a plurality of proximal ring openings, a means for securing an object in the proximal ring openings, a distal ring having a plurality of distal ring openings, a means for securing an object in the distal ring openings, and a plurality of rods each having a proximal end and a distal end. The proximal end of each of the plurality of rods is adapted to be received in one of the plurality of proximal ring openings, the distal end of each of the plurality of rods is adapted to be received in one of the plurality of distal ring openings, and at least one of the proximal ring and the distal ring is adjustably attached to the rods. The method includes adjusting the prosthetic device by moving the proximal ring and/or the distal ring relative to at least one of the rods.

### CROSS-REFERENCES TO RELATED APPLICATIONS/PATENTS

This application relates back to and claims the benefit of priority from U.S. Provisional Application for Patent No. 62/015,034 titled “Modular Forearm” and filed on Jun. 20, 2014.

### FIELD OF THE INVENTION

The present invention relates generally to prosthetic devices, and particularly to adjustable prosthetic devices.

### BACKGROUND AND DESCRIPTION OF THE PRIOR ART

It is known to use prosthetic devices to assist individuals lacking extremities or parts thereof. Conventional prosthetic devices, however, suffer from one or more disadvantages. For example, conventional prosthetic devices are difficult to adjust to the user's desired position. Conventional prosthetic devices are also expensive to adjust to the user's desired position. In addition, conventional prosthetic devices are undesirably heavy and rigid. Conventional prosthetic devices are also difficult and expensive to fabricate. Further, conventional prosthetic devices are cosmetically unattractive and do not sufficiently absorb external forces.

It would be desirable, therefore, if an apparatus and method for a prosthetic device could be provided that would be easy to adjust. It would also be desirable if such an apparatus and method for a prosthetic device could be provided that would be inexpensive to adjust. It would be further desirable if such an apparatus and method for a prosthetic device could be provided that would be light weight and flexible. It would be still further desirable if such an apparatus and method for a prosthetic device could be provided that would be easy and inexpensive to fabricate. In addition, it would be desirable if an apparatus and method for a prosthetic device could be provided that would be cosmetically attractive and sufficiently absorb external forces.

### ADVANTAGES OF THE PREFERRED EMBODIMENTS OF THE INVENTION

Accordingly, it is an advantage of the preferred embodiments of the invention described and claimed herein to provide an apparatus and method for a prosthetic device that is easy to adjust to the user's desired position. It is also an advantage of the preferred embodiments of the invention described and claimed herein to provide an apparatus and method for a prosthetic device that is inexpensive to adjust. It is another advantage of the preferred embodiments of the invention described and claimed herein to provide an apparatus and method for a prosthetic device that is light weight and flexible. It is a further advantage of the preferred embodiments of the invention described and claimed herein to provide an apparatus and method for a prosthetic device that is easy and inexpensive to fabricate. It is a still further advantage of the preferred embodiments of the invention described and claimed herein to provide an apparatus and method for a prosthetic device that is cosmetically attractive and absorbs external forces.

Additional advantages of the preferred embodiments of the invention will become apparent from an examination of the drawings and the ensuing description.

### SUMMARY OF THE INVENTION

The apparatus of the invention comprises a prosthetic device including a proximal ring having a plurality of proximal ring openings and being adapted to be mounted to a socket, a means for securing an object in the proximal ring openings, a distal ring having a plurality of distal ring openings and being spaced apart from the proximal ring, a means for securing an object in the distal ring openings, and a plurality of rods each having a proximal end and a distal end. In the preferred prosthetic device, the proximal end of each of the plurality of rods is adapted to be received in one of the plurality of proximal ring openings, the distal end of each of the plurality of rods is adapted to be received in one of the plurality of distal ring openings, and at least one of the proximal ring and the distal ring is adjustably attached to the plurality of rods.

The method of the invention comprises providing a prosthetic device which includes a proximal ring having a plurality of proximal ring openings and being adapted to be mounted to a socket, a means for securing an object in the proximal ring openings, a distal ring having a plurality of distal ring openings and being spaced apart from the proximal ring, a means for securing an object in the distal ring openings, and a plurality of rods each having a proximal end and a distal end. In the preferred prosthetic device, the proximal end of each of the plurality of rods is adapted to be received in one of the plurality of proximal ring openings, the distal end of each of the plurality of rods is adapted to be received in one of the plurality of distal ring openings, and at least one of the proximal ring and the distal ring is adjustably attached to the plurality of rods. The preferred method also comprises adjusting the prosthetic device by moving at least one of the proximal ring and the distal ring relative to at least one of the plurality of rods.

### BRIEF DESCRIPTION OF THE DRAWINGS

The presently preferred embodiments of the invention are illustrated in the accompanying drawings, in which like reference numerals represent like parts throughout, and in which:

FIG. 1 is a perspective view of the preferred embodiment of the prosthetic device in accordance with the present invention.

FIG. 2 is an exploded perspective view of the preferred prosthetic device illustrated in FIG. 1.

FIG. 3 is a front (lateral) view of the preferred prosthetic device illustrated in FIGS. 1-2.

FIG. 4 is a top view of the preferred prosthetic device illustrated in FIGS. 1-3.

FIG. 5 is a left end (anterior) view of the preferred prosthetic device illustrated in FIGS. 1-4.

FIG. 6 is a perspective view of a first alternative embodiment of the prosthetic device in accordance with the present invention.

FIG. 7 is an exploded view of the preferred prosthetic device illustrated in FIG. 6.

FIG. 8 is a front (lateral) view of the preferred prosthetic device illustrated in FIGS. 6-7.

FIG. 9 is a top view of the preferred prosthetic device illustrated in FIGS. 6-8.

FIG. 10 is a bottom view of the preferred prosthetic device illustrated in FIGS. 6-9.

FIG. 11 is a front (lateral) view of the preferred prosthetic device illustrated in FIGS. 6-10 with an exemplary wrist unit and terminal device

FIG. 12 is a front (lateral) view of a second alternative embodiment of the prosthetic device in accordance with the present invention.

FIG. 13 is a right end (posterior) view of the second alternative embodiment of the prosthetic device illustrated in FIG. 12.

### DESCRIPTION OF THE PREFERRED EMBODIMENTS OF THE INVENTION

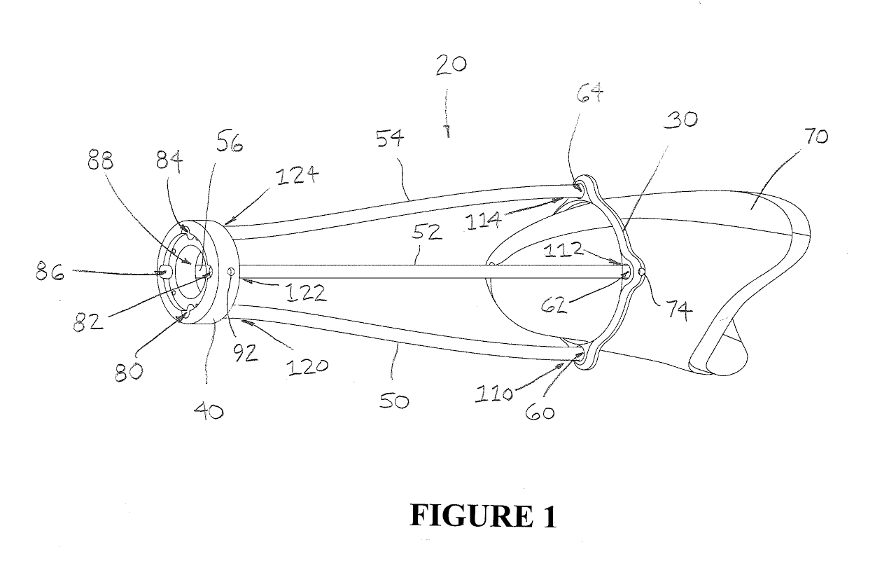

Referring now to the drawings, the preferred embodiments of the prosthetic device in accordance with the present invention are illustrated by FIGS. 1 through 13. As shown in FIGS. 1-13, the preferred embodiments of the invention described and claimed herein are adapted to provide an apparatus and method for a prosthetic device that is easy to adjust to the user's desired position. The preferred embodiments of the invention described and claimed herein are also adapted to provide an apparatus and method for a prosthetic device that is inexpensive to adjust. The preferred embodiments of the invention described and claimed herein are further adapted to provide an apparatus and method for a prosthetic device that is light weight and flexible. The preferred embodiments of the invention described and claimed herein are still further adapted to provide an apparatus and method for a prosthetic device that is easy and inexpensive to fabricate. In addition, the preferred embodiments of the invention described and claimed herein are adapted to provide an apparatus and method for a prosthetic device that is cosmetically attractive and absorbs external forces.

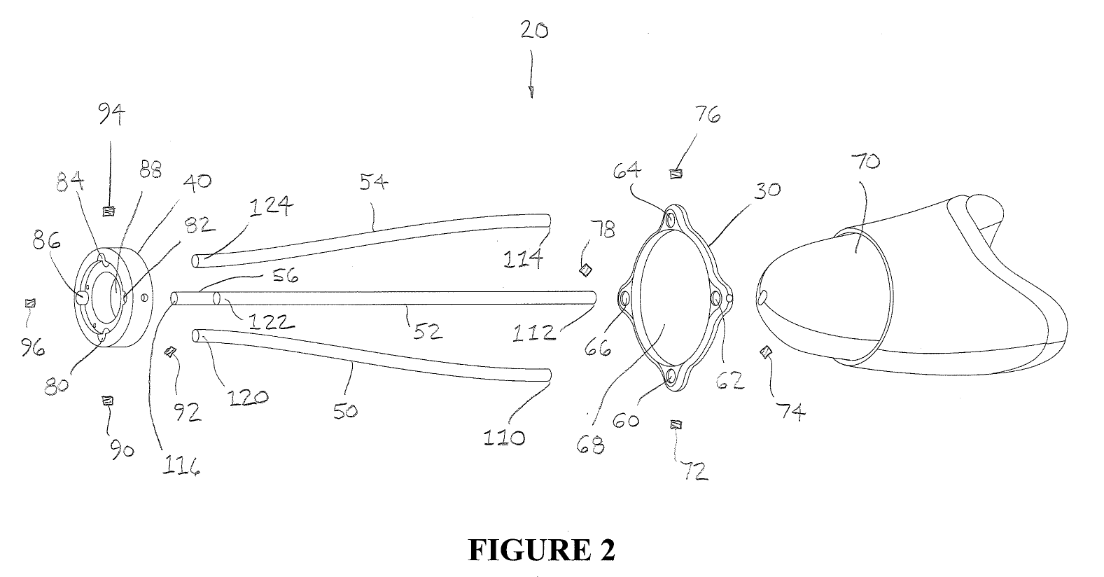

Referring now to FIG. 1, a perspective view of the preferred embodiment of the prosthetic device in accordance with the present invention is illustrated. As shown in FIG. 1, the preferred prosthetic device is designated generally by reference numeral 20. Preferred prosthetic device 20 comprises proximal ring 30, distal ring 40, and a plurality of rods 50, 52, 54, and 56. Preferred proximal ring 30 comprises a plurality of proximal ring openings 60, 62, 64, 66 (see FIGS. 2), and 68 (see FIG. 2) and is adapted to be mounted to exemplary socket 70. More particularly, exemplary socket 70 is adapted to be mounted into proximal ring opening 68. Preferably, proximal ring 30 is fixedly mounted to exemplary socket 70 by laminating it to the socket with carbon fiber, but it is contemplated within the scope of the invention that the proximal ring may be attached to the socket by vacuum forming plastic over the ring, gluing it to the socket, and the like. It is also contemplated within the scope of the invention that the proximal ring may be removably mounted to a socket. It is also contemplated within the scope of the invention that the proximal ring may be mounted to a joint unit such as an elbow unit or a knee unit.

Still referring to FIG. 1, preferred proximal ring 30 also comprises a means for securing an object in proximal ring openings 60, 62, 64, and 66 such as plurality of proximal ring set screws 72, 74, 76, and 78. Each of the preferred plurality of proximal ring set screws 72 (see FIG. 2), 74, 76 (see FIGS. 2), and 78 (see FIG. 2) is adapted to be in operable communication with one of the plurality of proximal ring openings 60, 62, 64, and 66. While FIG. 1 illustrates the preferred configuration and arrangement of the proximal ring, it is contemplated within the scope of the invention that the proximal ring may be of any suitable configuration and arrangement. It is also contemplated within the scope of the invention that the proximal ring may comprise fewer or more than five proximal ring openings. It is further contemplated within the scope of the invention that any suitable device, mechanism, assembly, or combination thereof may be used to adjustably secure a rod in a proximal ring opening such as pins, bolts, screws, shark bite fittings, and the like. It is still further contemplated within the scope of the invention that a rod may be fixedly retained in a proximal ring opening such as by a polyurethane adhesive that can be heated for removal and the like.

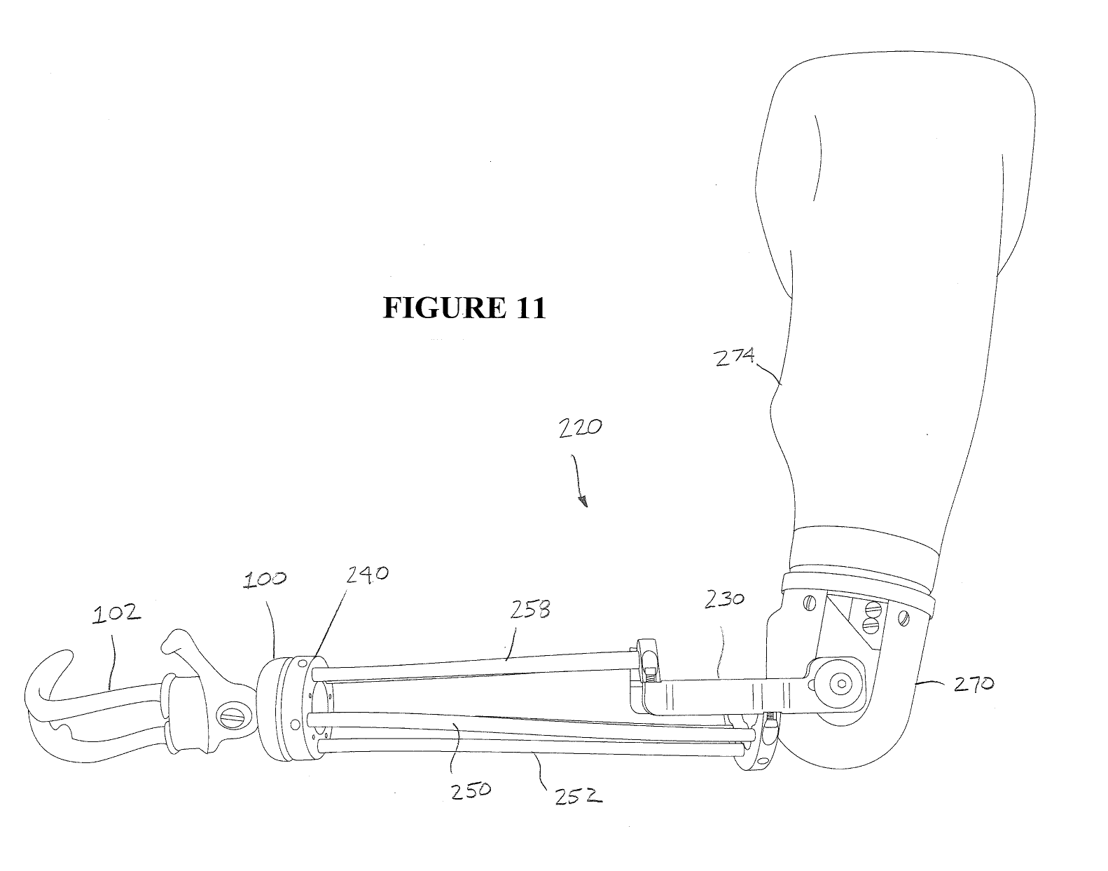

Still referring to FIG. 1, preferred distal ring 40 comprises a plurality of distal ring openings 80, 82, 84, 86, and 88 and is spaced apart from proximal ring 30. In addition, preferred distal ring 40 comprises a means for securing an object in the plurality of distal ring openings such as a plurality of distal ring set screws 90 (see FIG. 2), 92, 94 (see FIGS. 2), and 96 (see FIG. 2). Each of the preferred plurality of distal ring set screws 90, 92, 94, and 96 is adapted to be in operable communication with one of the plurality of distal ring openings 80, 82, 84, and 86. Preferred distal ring 40 is also adapted to receive a connecting device such as wrist unit 100 (see FIG. 11) that is adapted to removably receive a terminal device such as claw 102 (see FIG. 11). It is contemplated within the scope of the invention that wrist unit 100 may be any suitable prosthetic connecting device that may be modified or adapted to attach to distal ring 40. While FIG. 1 illustrates the preferred configuration and arrangement of the distal ring, it is contemplated within the scope of the invention that the distal ring may be of any suitable configuration and arrangement. It is also contemplated within the scope of the invention that the distal ring may comprise fewer or more than five distal ring openings. It is further contemplated within the scope of the invention that any suitable device, mechanism, assembly, or combination thereof may be used to adjustably secure a rod in a distal ring opening such as pins, bolts, screws, shark bite fittings, and the like. It is still further contemplated within the scope of the invention that a rod may be fixedly retained in a distal ring opening such as by a polyurethane adhesive that may be heated for removal and the like. In addition, it is contemplated within the scope of the invention that the distal ring is adapted to removably receive a prosthetic ankle and/or foot device.

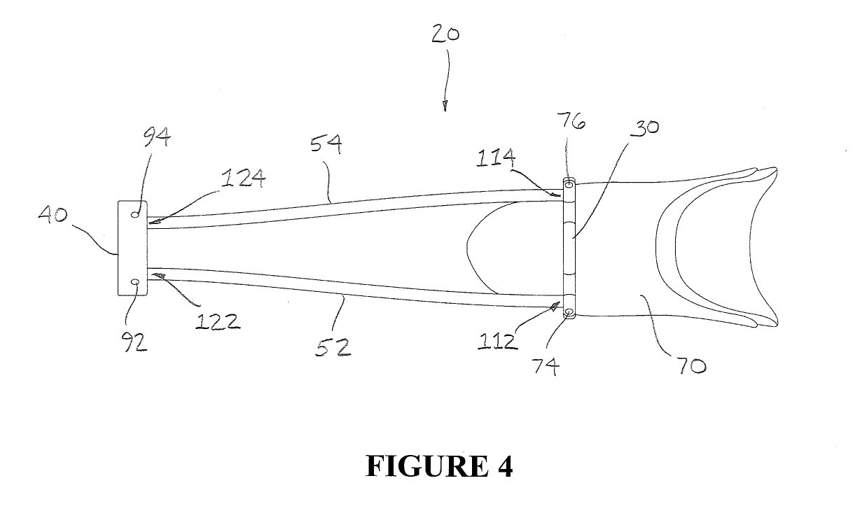

Still referring to FIG. 1, preferred plurality of rods 50, 52, 54, and 56 each comprise a proximal end 110, 112, 114, and 116 (see FIG. 2), respectively, and a distal end 120, 122, 124, and 126 (see FIG. 4), respectively. Preferred proximal ends 110, 112, 114, and 116 are each adapted to be received in one of the plurality of proximal ring openings 60, 62, 64, and 66. Similarly, preferred distal ends 120, 122, 124, and 126 are each adapted to be received in one of the plurality of distal ring openings 80, 82, 84, and 86. In preferred prosthetic device 20, at least one of proximal ring 30 and distal ring 40 is adjustably attached to the plurality of rods 50, 52, 54, and 56. Preferably, each of the plurality of rods 50, 52, 54, and 56 is resilient, light weight, and adapted to absorb forces. In addition, each of the preferred plurality of rods 50, 52, 54, and 56 comprises a thermoplastic material such as polyether ether ketone. While FIG. 1 illustrates the preferred configuration and arrangement of the plurality of rods, it is contemplated within the scope of the invention that the plurality of rods may be of any suitable configuration and arrangement. More particularly, it is contemplated within the scope of the invention that one or more of the plurality of rods has a cross-sectional shape that is semi-circular, square, rectangular, or any other suitable shape. It is also contemplated within the scope of the invention that the plurality of rods may comprise fewer or more than four rods. It is further contemplated within the scope of the invention that one or more of the plurality of rods may comprise a carbon/thermoplastic composite.

Referring now to FIG. 2, an exploded perspective view of preferred prosthetic device 20 is illustrated. As shown in FIG. 2, preferred prosthetic device 20 comprises proximal ring 30, distal ring 40, rods 50, 52, 54, and 56, proximal ring openings 60, 62, 64, 66, and 68, socket 70, proximal ring set screws 72, 74, 76, and 78, distal ring openings 80, 82, 84, 86, and 88, distal ring set screws 90, 92, 94, and 96, rod proximal ends 110, 112, 114, and 116, and rod distal ends 120, 122, 124, and 126.

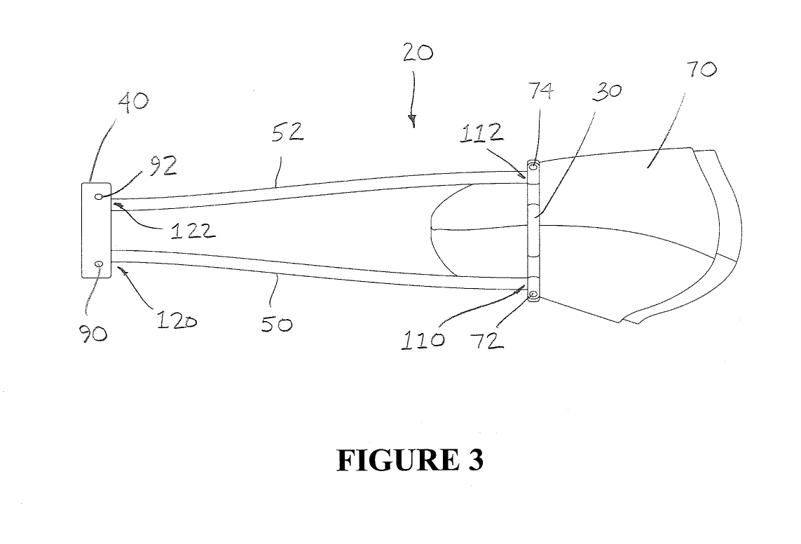

Referring now to FIG. 3, a front (lateral) view of preferred prosthetic device 20 is illustrated. As shown in FIG. 3, preferred prosthetic device 20 comprises proximal ring 30, distal ring 40, rods 50 and 52, socket 70, proximal ring set screws 72 and 74, distal ring set screws 90 and 92, rod proximal ends 110 and 112, and rod distal ends 120 and 122.

Referring now to FIG. 4, a top view of preferred prosthetic device 20 is illustrated. As shown in FIG. 4, preferred prosthetic device 20 comprises proximal ring 30, distal ring 40, rods 52 and 54, socket 70, proximal ring set screws 74 and 76, distal ring set screws 92 and 94, rod proximal ends 112 and 114, and rod distal ends 122 and 124.

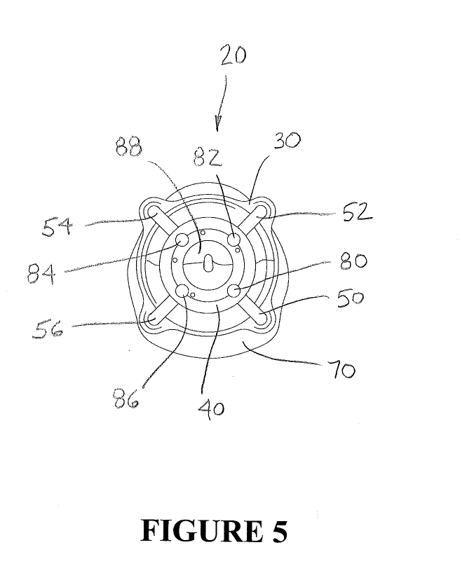

Referring now to FIG. 5, a left end view of preferred prosthetic device 20 is illustrated. As shown in FIG. 5, preferred prosthetic device 20 comprises proximal ring 30, distal ring 40, rods 50, 52, 54, and 56, socket 70, and distal ring openings 80, 82, 84, 86, and 88.

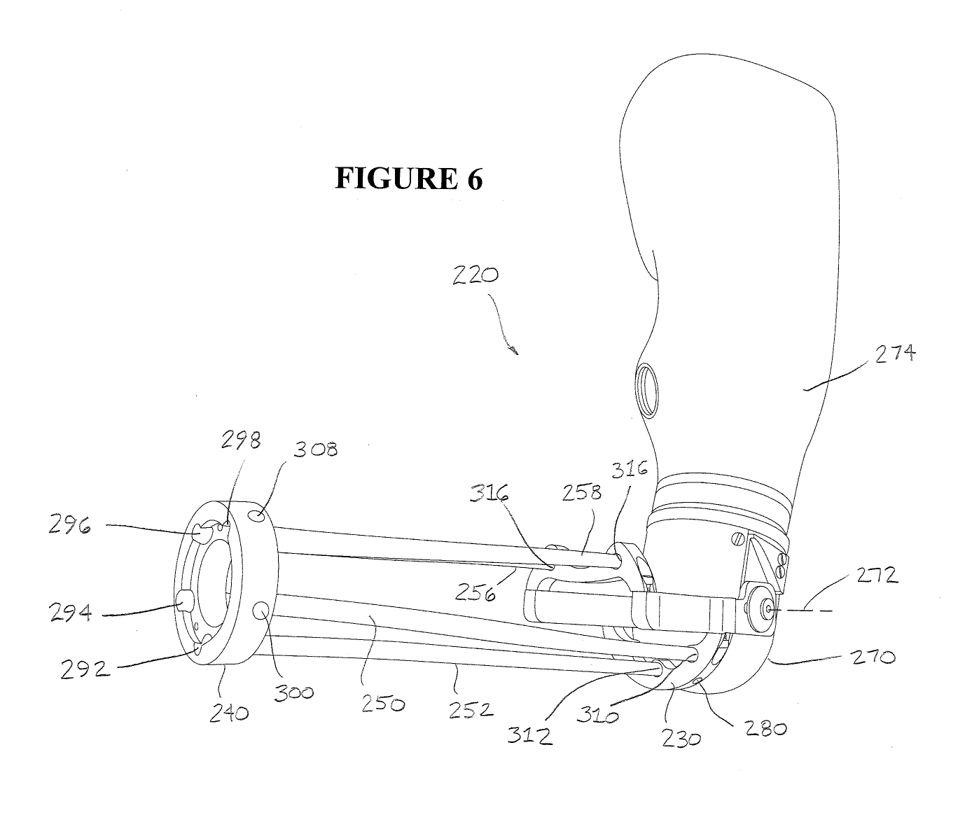

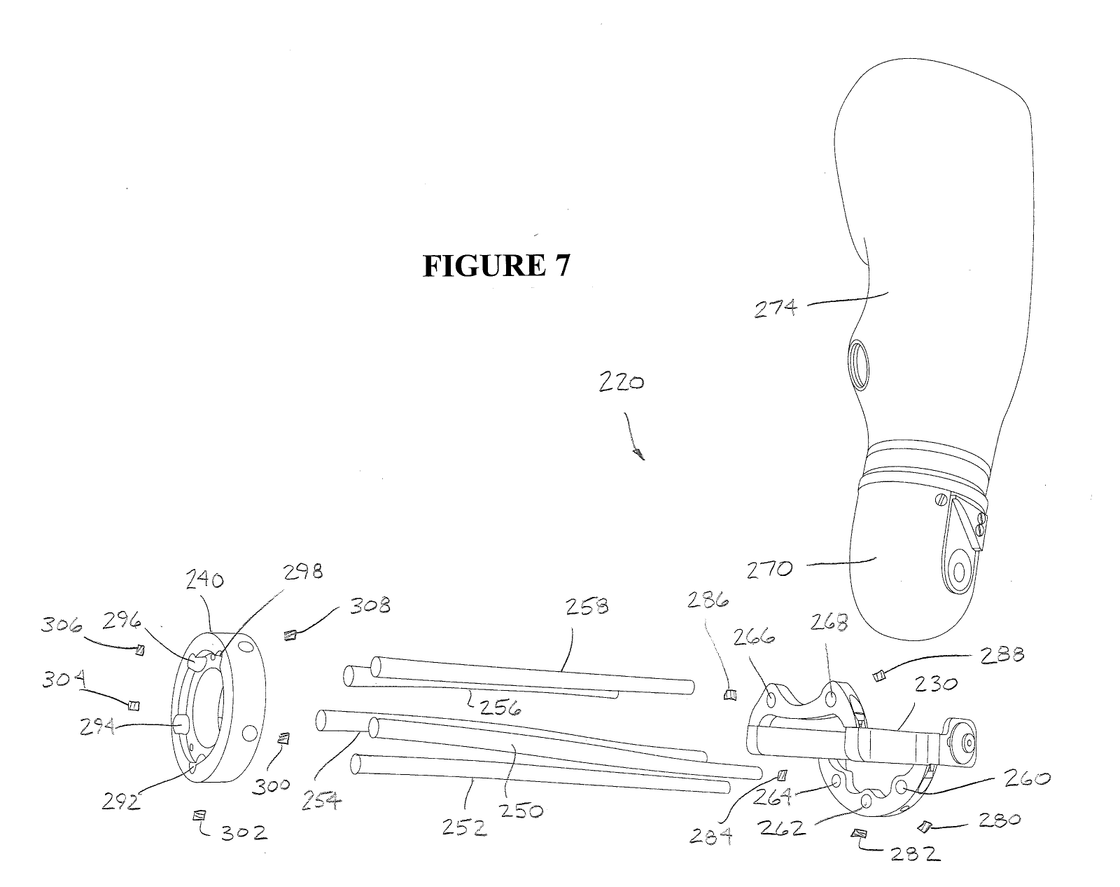

Referring now to FIG. 6, a perspective view of a first alternative embodiment of the prosthetic device in accordance with the present invention is illustrated. As shown in FIG. 6, the first alternative embodiment of the prosthetic device is designated generally by reference numeral 220. Preferred prosthetic device 220 comprises modified proximal ring 230, distal ring 240, and a plurality of rods 250, 252, 254, 256, and 258. Preferred modified proximal ring 230 comprises a plurality of modified proximal ring openings 260, 262, 264, 266, and 268 (see FIG. 7) and is adapted to be mounted to a joint unit such as exemplary elbow unit 270. More particularly, exemplary elbow unit 270 is adapted to be pivotally mounted to modified proximal ring 230. Preferably, modified proximal ring 230 is pivotally mounted to elbow unit 270 at elbow axis 272. Preferred elbow unit 270 is fixedly mounted to exemplary socket 274 by laminating it to the socket with carbon fiber, but it is contemplated within the scope of the invention that the elbow unit may be fixedly mounted to the socket by vacuum forming plastic over it, adhesives, and the like. It is also contemplated within the scope of the invention that the elbow unit may be removably mounted to a socket.

Still referring to FIG. 6, preferred modified proximal ring 230 comprises a plurality of modified proximal ring set screws 280, 282 (see FIG. 7), 284 (see FIG. 7), 286 (see FIGS. 7), and 288. Each of the preferred plurality of modified proximal ring set screws 280, 282, 284, 286, and 288 is adapted to be in operable communication with one of the plurality of modified proximal ring openings 260, 262, 264, 266, and 268. While FIG. 1 illustrates the preferred configuration and arrangement of the modified proximal ring, it is contemplated within the scope of the invention that the modified proximal ring may be of any suitable configuration and arrangement. It is also contemplated within the scope of the invention that the modified proximal ring may comprise fewer or more than five modified proximal ring openings. It is further contemplated within the scope of the invention that any suitable device, mechanism, assembly, or combination thereof may be used to adjustably secure a rod in a modified proximal ring opening such as pins, bolts, screws, shark bite fittings, and the like. It is still further contemplated within the scope of the invention that a rod may be fixedly retained in a modified proximal ring opening such as by an adhesive that can be heated for removal and the like. It is also contemplated within the scope of the invention that the elbow unit may be of any suitable configuration and arrangement. In addition, it is contemplated within the scope of the invention that preferred prosthetic device 220 may comprise a knee unit to which modified proximal ring 230 is mounted instead of elbow unit 270.

Still referring to FIG. 6, preferred distal ring 240 comprises a plurality of distal ring openings 290, 292, 294, 296, and 298 and is spaced apart from modified proximal ring 230. In addition, preferred distal ring 240 comprises a plurality of distal ring set screws 300, 302, 304, 306, 308. Each of the plurality of distal ring set screws 300, 302, 304, 306, and 308 is adapted to be in operable communication with one of the plurality of distal ring openings 290, 292, 294, 296, and 298. Preferred distal ring 240 is also adapted to receive wrist unit 100 (see FIG. 11) that is adapted to removably receive claw 102 (see FIG. 11). It is contemplated within the scope of the invention that wrist unit 100 may be any suitable prosthetic connecting device that may be modified or adapted to attach to distal ring 40. While FIG. 6 illustrates the preferred configuration and arrangement of the distal ring, it is contemplated within the scope of the invention that the distal ring may be of any suitable configuration and arrangement. It is also contemplated within the scope of the invention that the distal ring may comprise fewer or more than five distal ring openings. It is further contemplated within the scope of the invention that any suitable device, mechanism, assembly, or combination thereof may be used to adjustably secure a rod in a distal ring opening such as pins, bolts, screws, shark bite fittings, and the like. It is still further contemplated within the scope of the invention that a rod may be fixedly retained in a distal ring opening such as by an adhesive that may be heated for removal and the like. In addition, it is contemplated within the scope of the invention that the distal ring is adapted to removably receive a prosthetic ankle and/or foot device.

Still referring to FIG. 6, preferred plurality of rods 250, 252, 254, 256, and 258 each comprise a proximal end 310, 312, 314, 316, and 318 respectively, and a distal end 320, 322, 324, 326, and 328 (see FIG. 7), respectively. Preferred proximal ends 310, 312, 314, 316, and 318 are each adapted to be received in one of the plurality of modified proximal ring openings 260, 262, 264, 266, and 268. Similarly, preferred distal ends 320, 322, 324, 326, and 328 are each adapted to be received in one of the plurality of distal ring openings 280, 282, 284, 286, and 288. In preferred prosthetic device 220, at least one of modified proximal ring 230 and distal ring 240 is adjustably attached to the plurality of rods 250, 252, 254, 256, and 258. Preferably, each of the plurality of rods 250, 252, 254, 256, and 258 is resilient, light weight, and adapted to absorb forces. In addition, each of the preferred plurality of rods 250, 252, 254, 256, and 258 comprises a thermoplastic material such as polyether ether ketone. While FIG. 6 illustrates the preferred configuration and arrangement of the plurality of rods, it is contemplated within the scope of the invention that the plurality of rods may be of any suitable configuration and arrangement. It is also contemplated within the scope of the invention that the plurality of rods may comprise fewer or more than four rods.

Referring now to FIG. 7, an exploded view of preferred prosthetic device 220 is illustrated. As shown in FIG. 7, preferred prosthetic device 220 comprises modified proximal ring 230, distal ring 240, a plurality of rods 250, 252, 254, 256, and 258 modified proximal ring openings 260, 262, 264, 266, and 268, elbow unit 270, socket 274, modified proximal ring set screws 280, 282, 284, 286, and 288 distal ring openings 290, 292, 294, 296, and 298, distal ring set screws 300, 302, 304, 306, and 308 rod proximal ends 310, 312, 314, 316, and 318, and rod distal ends 320, 322, 324, 326, and 328.

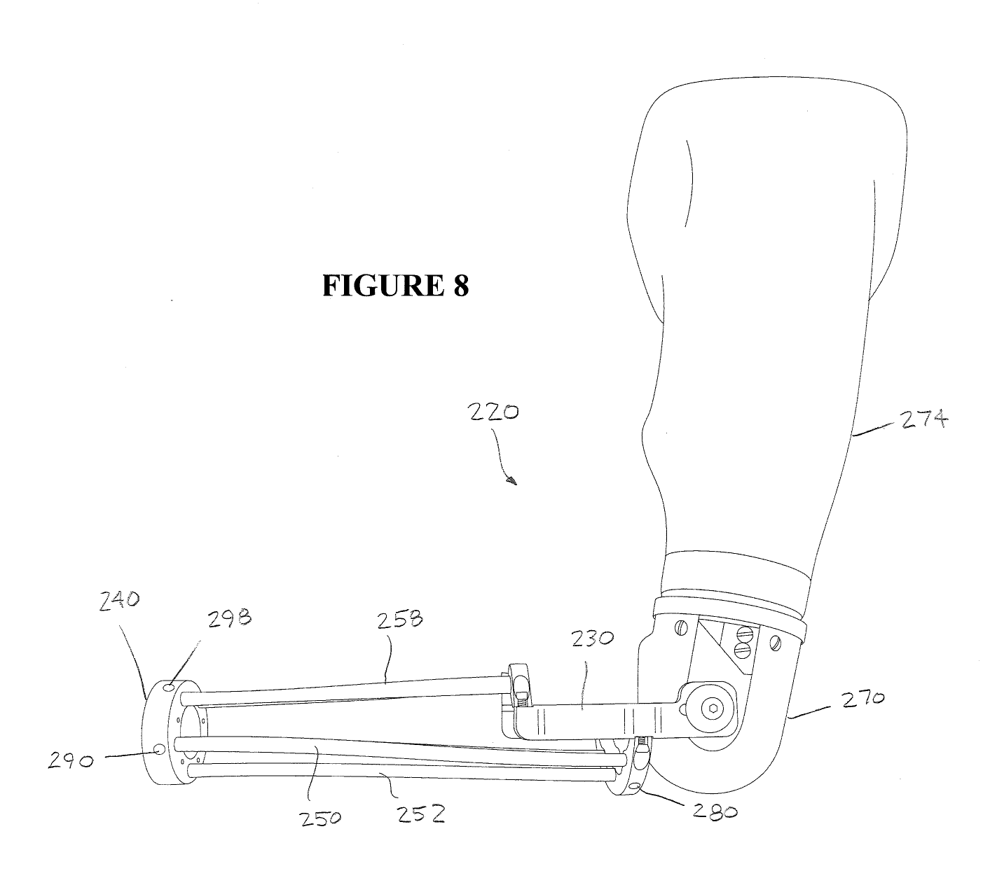

Referring now to FIG. 8, a front (lateral) view of preferred prosthetic device 220 is illustrated, As shown in FIG. 8, preferred prosthetic device 220 comprises modified proximal ring 230, distal ring 240, rods 250, 252, and 258, elbow unit 270, socket 274, modified proximal ring set screws 280, and distal ring set screws 290 and 298.

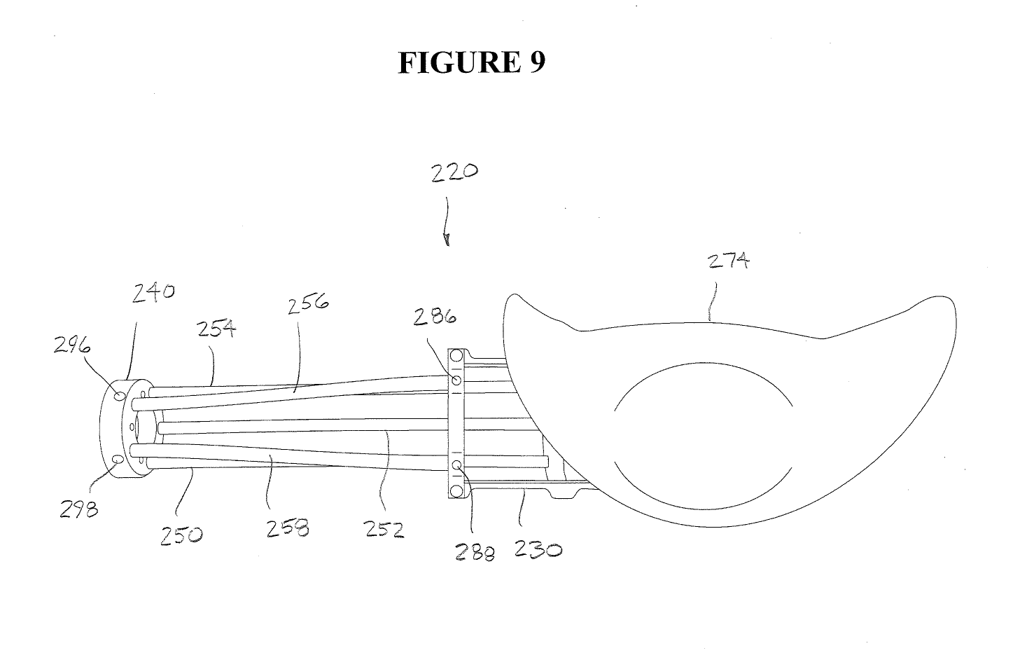

Referring now to FIG. 9, a top view of preferred prosthetic device 220 is illustrated. As shown in FIG. 9, preferred prosthetic device 220 comprises modified proximal ring 230, distal ring 240, rods 250, 252, 254, 256, and 258, elbow unit 270, socket 274, modified proximal ring set screws 286 and 288, and distal ring set screws 296 and 298.

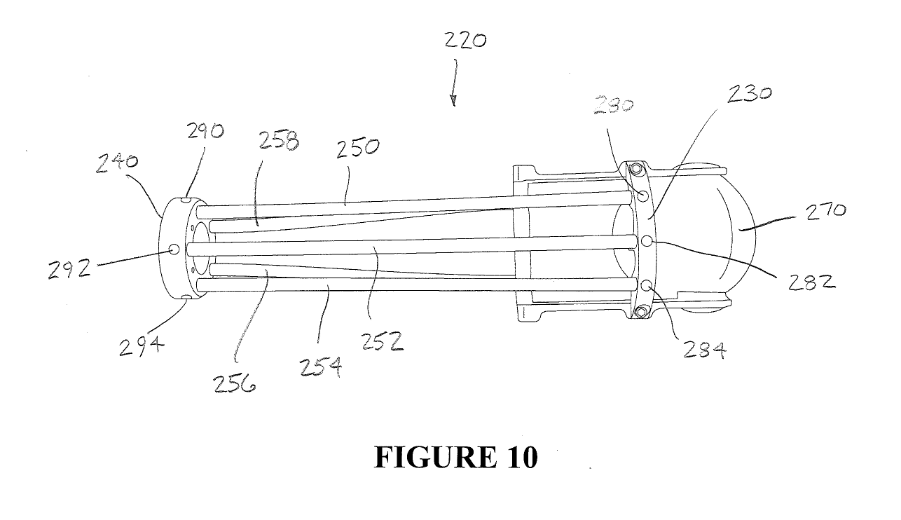

Referring now to FIG. 10, a bottom view of the preferred prosthetic device 220 is illustrated. As shown in FIG. 10, preferred prosthetic device 220 comprises modified proximal ring 230, distal ring 240, rods 250, 252, 254, 256, and 258, elbow unit 270, modified proximal ring set screws 280, 282, and 284, and distal ring set screws 290, 292 and 294.

Referring now to FIG. 11, a front (lateral) view of preferred prosthetic device 220 is illustrated with exemplary wrist unit 100 and claw 102. As shown in FIG. 11, preferred prosthetic device 220 comprises modified proximal ring 230, distal ring 240, rods 250, 252, and 258, elbow unit 270, and socket 274.

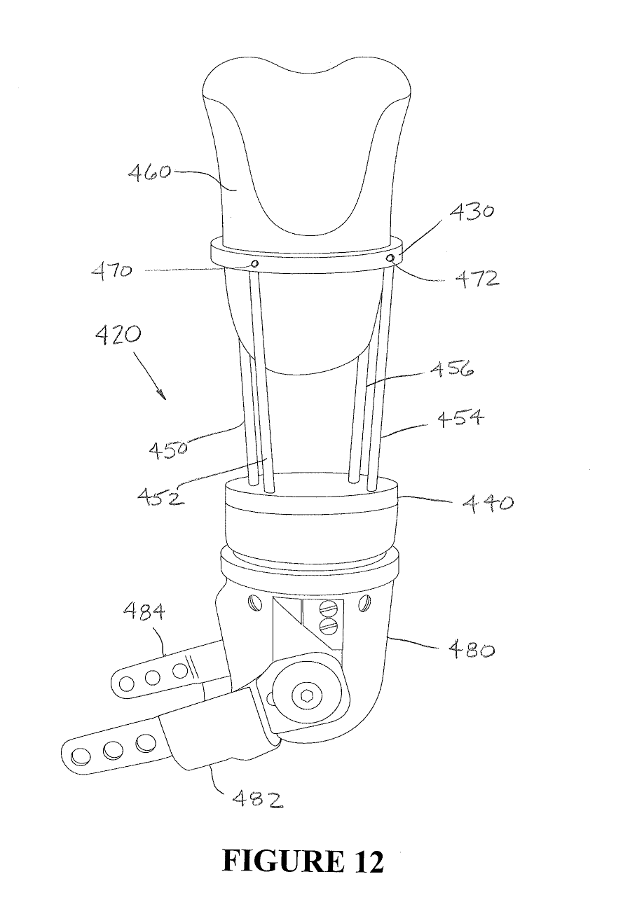

Referring now to FIG. 12, a perspective view of a second alternative embodiment of the prosthetic device in accordance with the present invention is illustrated. As shown in FIG. 12, the second alternative embodiment of the prosthetic device in accordance with the present invention is designated generally by reference numeral 420. Preferred prosthetic device 420 comprises a first proximal ring such as upper limb proximal ring 430, a first distal ring such as upper limb distal ring 440, and a plurality of upper limb rods 450, 452, 454, and 456. Upper limb proximal ring 430 comprises a plurality of upper limb proximal ring openings each of which is adapted to receive the proximal end of one of the plurality of upper limb rods 450, 452, 454, and 456. Each of the proximal ends of the plurality of upper limb rods is preferably fixedly mounted in the upper limb proximal ring, but it is contemplated within the scope of the invention that one or more of the proximal ends of the plurality of upper limb rods may be adjustable mounted in the upper limb proximal ring. In addition, upper limb proximal ring 430 is also adapted to be mounted on socket 460. While upper limb proximal ring 430 is preferably fixedly mounted to socket 460, it is contemplated within the scope of the invention that the upper limb proximal ring may be removably mounted to socket 460.

Still referring to FIG. 12, preferred upper limb distal ring 440 also comprises a plurality of upper limb distal ring openings each of which is adapted to receive the distal end of one of the plurality of upper limb rods. Preferably, upper limb distal ring 440 also comprises a means for securing the distal end of at least one of the upper limb rods such as set screws 470 and 472. While set screws 470 and 472 are the preferred means for securing the distal ends of the plurality of upper limb rods, it is contemplated within the scope of the invention that one or more distal ends of the upper limb rods may be removably secured in upper limb distal ring 440 with pins, bolts, screws, shark bite fittings, and the like. It is also contemplated within the scope of the invention that one or more of the distal ends of the plurality of upper limb rods may be fixedly attached to the upper limb distal ring by adhesives and the like. Preferably, upper limb distal ring 440 is adapted to be connected to a joint unit such as exemplary elbow unit 480 having attachment arms 482 and 484. Further, preferred prosthetic device 420 is adapted to be provided with a lower limb prosthetic device as described hereinabove and illustrated in FIGS. 6-11. While FIG. 12 illustrates the preferred configuration and arrangement of the upper limb proximal ring, the upper limb distal ring, and the plurality of upper limb rods, it is contemplated within the scope of the invention that the upper limb proximal ring, the upper limb distal ring, and the plurality of upper limb rods may be of any suitable configuration and arrangement.

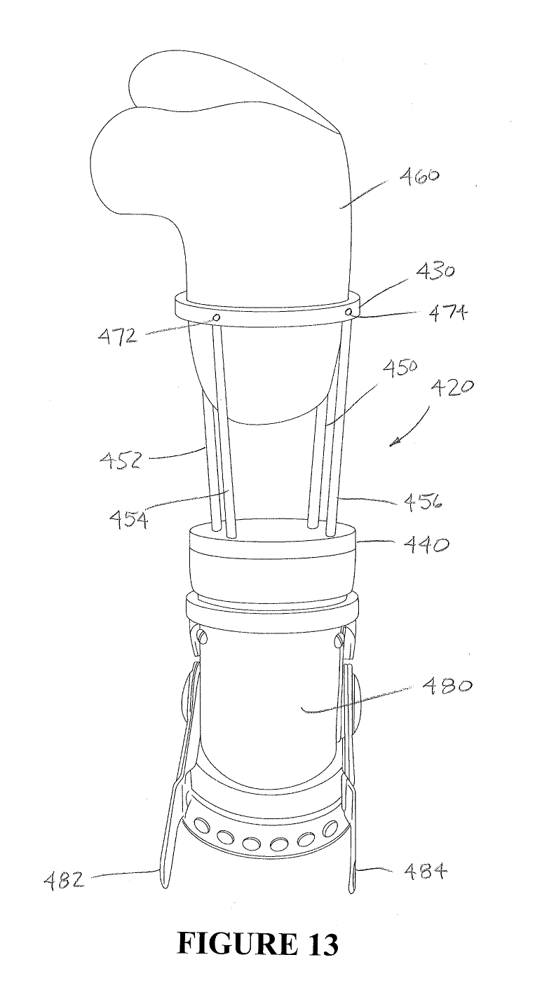

Referring now to FIG. 13, a back (posterior) view of preferred prosthetic device 420 is illustrated. As shown in FIG. 13, preferred prosthetic device 420 comprises upper limb proximal ring 430, upper limb distal ring 440, a plurality of upper limb rods 450, 452, 454, and 456, socket 460, set screws 472 and 474, and exemplary elbow unit 480 having attachment arms 482 and 484. While FIG. 13 illustrates preferred prosthetic device 420 which is adapted for use on an upper extremity, it is contemplated within the scope of the invention that the prosthetic device may be adapted for use on a lower extremity with a knee unit instead of an elbow unit.

The invention also comprises a method for adjusting a prosthetic device. The preferred method comprises providing a prosthetic device. The preferred prosthetic device comprises a proximal ring having a plurality of proximal ring openings and being adapted to be mounted to a socket, a means for securing an object in the proximal ring openings, a distal ring having a plurality of distal ring openings and being spaced apart from the proximal ring, a means for securing an object in the distal ring openings, and a plurality of rods, each of which has a proximal end and a distal end. In the preferred embodiments of the prosthetic device, the proximal end of each of the plurality of rods is adapted to be received in one of the plurality of proximal ring openings, the distal end of each of the plurality of rods is adapted to be received in one of the plurality of distal ring openings, and at least one of the proximal ring and the distal ring is adjustably attached to the plurality of rods. The preferred method also comprises adjusting the prosthetic device by moving at least one of the proximal ring and the distal ring relative to at least one of the plurality of rods. Also in the preferred methods, at least one of the plurality of rods comprises a thermoplastic material such as polyether ether ketone.

In operation, several advantages of the preferred embodiments of the apparatus and method for a prosthetic device are achieved. For example, the preferred embodiments of the invention described and claimed herein are adapted to provide an apparatus and method for a prosthetic device that is easy to adjust to the user's desired position. The preferred embodiments of the invention described and claimed herein are also adapted to provide an apparatus and method for a prosthetic device that is inexpensive to adjust. The preferred embodiments of the invention described and claimed herein are further adapted to provide an apparatus and method for a prosthetic device that is light weight and flexible. The preferred embodiments of the invention described and claimed herein are still further adapted to provide an apparatus and method for a prosthetic device that is easy and inexpensive to fabricate. In addition, the preferred embodiments of the invention described and claimed herein are adapted to provide an apparatus and method for a prosthetic device that is cosmetically attractive and absorbs external forces.

Although this description contains many specifics, these should not be construed as limiting the scope of the invention but as merely providing illustrations of some of the presently preferred embodiments thereof, as well as the best mode contemplated by the inventors of carrying out the invention. The invention, as described herein, is susceptible to various modifications and adaptations, and the same are intended to be comprehended within the meaning and range of equivalents of the appended claims.

What is claimed is:

1. A prosthetic device, said prosthetic device comprising:
(a) a proximal ring, said proximal ring having a plurality of proximal ring openings and being adapted to be mounted to a socket;
(b) a means for securing an object in the proximal ring openings;
(c) a distal ring, said distal ring having a plurality of distal ring openings and being spaced apart from the proximal ring;
(d) a means for securing an object in the distal ring openings;
(e) a plurality of rods, each of said plurality of rods having a proximal end and a distal end;
wherein the proximal end of each of the plurality of rods is adapted to be received in one of the plurality of proximal ring openings; and wherein the distal end of each of the plurality of rods is adapted to be received in one of the plurality of distal ring openings; and wherein at least one of the proximal ring and the distal ring is adjustably attached to the plurality of rods.

2. The prosthetic device of claim 1 wherein the proximal ring comprises a plurality of proximal ring set screws, each of said plurality of proximal ring set screws in operable communication with one of the plurality of proximal ring openings.

3. The prosthetic device of claim 1 wherein the distal ring comprises a plurality of distal ring set screws, each of said plurality of distal ring set screws in operable communication with one of the plurality of distal ring openings.

4. The prosthetic device of claim 1 wherein the distal ring is adapted to receive a wrist unit.

5. The prosthetic device of claim 4 wherein the wrist unit is adapted to removably receive a terminal device.

6. The prosthetic device of claim 4 wherein the wrist unit is adapted to be removably received by the distal ring.

7. The prosthetic device of claim 1 wherein at least one of the plurality of rods is resilient.

8. The prosthetic device of claim 1 wherein at least one of the plurality of rods is light weight.

9. The prosthetic device of claim 1 wherein at least one of the plurality of rods is adapted to absorb forces.

10. The prosthetic device of claim 1 wherein at least one of the plurality of rods comprises a thermoplastic material.

11. The prosthetic device of claim 1 wherein at least one of the plurality of rods comprises polyether ether ketone.

12. The prosthetic device of claim 1 wherein the means for securing an object in the proximal ring openings is a set screw.

13. The prosthetic device of claim 1 wherein the means for securing an object in the distal ring openings is a set screw.

14. The prosthetic device of claim 1 further comprising a joint unit.

15. A prosthetic device, said prosthetic device comprising:
(a) a first proximal ring, said first proximal ring having a plurality of first proximal ring openings and being adapted to be mounted to a socket;
(b) a means for securing an object in the first proximal ring openings;
(c) a first distal ring, said first distal ring having a plurality of first distal ring openings and being adapted to receive a joint unit;
(d) a means for securing an object in the first distal ring openings;
(e) a plurality of first rods, each of said plurality of first rods having a first rod proximal end and a first rod distal end;
(f) a joint unit;
(g) a second proximal ring, said second proximal ring having a plurality of second proximal ring openings and being adapted to be mounted to the elbow adapter;
(h) a means for securing an object in the second proximal ring openings;
(i) a second distal ring, said second distal ring having a plurality of second distal ring openings and being adapted to receive a wrist adapter;
(j) means for securing an object in the second distal ring openings;
(k) a plurality of second rods, each of said plurality of second rods having a second rod proximal end and a second rod distal end;
wherein the first rod proximal end of each of the plurality of first rods is adapted to be received in one of the plurality of first proximal ring openings; and wherein the first rod distal end of each of the plurality of first rods is adapted to be received in one of the plurality of first distal ring openings; and wherein the second rod proximal end of each of the plurality of second rods is adapted to be received in one of the plurality of second proximal ring openings; and wherein the second rod distal end of each of the plurality of second rods is adapted to be received in one of the plurality of second distal ring openings; and wherein at least one of the second proximal ring and the second distal ring is adjustably attached to the plurality of second rods.

16. The prosthetic device of claim 1 wherein at least one of the plurality of rods comprises a thermoplastic material.

17. The prosthetic device of claim 1 wherein at least one of the plurality of rods comprises polyether ether ketone.

18. A method for adjusting a prosthetic device, said method comprising:
(a) providing a prosthetic device, said prosthetic device comprising:
(i) a proximal ring, said proximal ring having a plurality of proximal ring openings and being adapted to be mounted to a socket;
(ii) a means for securing an object in the proximal ring openings;
(iii) a distal ring, said distal ring having a plurality of distal ring openings and being spaced apart from the proximal ring;
(iv) a means for securing an object in the distal ring openings;
(v) a plurality of rods, each of said plurality of rods having a proximal end and a distal end;
wherein the proximal end of each of the plurality of rods is adapted to be received in one of the plurality of proximal ring openings; and wherein the distal end of each of the plurality of rods is adapted to be received in one of the plurality of distal ring openings; and wherein at least one of the proximal ring and the distal ring is adjustably attached to the plurality of rods;
(b) adjusting the prosthetic device by moving at least one of the proximal ring and the distal ring relative to at least one of the plurality of rods.

19. The method of claim 18 wherein at least one of the plurality of rods comprises a thermoplastic material.

20. The method of claim 18 wherein at least one of the plurality of rods comprises polyether ether ketone.
